(19) United States
(12) Patent Application Publication
(10) Pub. No.: US 2011/0308286 A1
(43) Pub. Date: Dec. 22, 2011

(54) HOUSEHOLD COMPOST RECEPTACLE HAVING A CONE SHAPED OPENING

(76) Inventor: Ross Lyell Cowie, Ottawa (CA)

(21) Appl. No.: 12/820,308

(22) Filed: Jun. 22, 2010

(51) Int. Cl. C05F 9/04; C12M 1/16

(52) U.S. Cl. 71/14; 435/290.1

(57) ABSTRACT

Disclosed is a combination of a compost receptacle and removable newspaper liner. The compost receptacle defines an opening therein. Preferably the opening comprises two side walls that join together at two wall angles on opposite ends to form a flattened cone shape, the side walls tapering into a point. The newspaper liner is arranged into a shape that is complementary to the shape of the opening. Preferably the liner has side surfaces that meet at opposite angle portions and that taper into a closed point so that when the opening receives the newspaper liner, the two wall angles and the point self-align the newspaper liner therein during use to form a disposable compost collector out of the newspaper liner. In this way, in a related disclosed method, the compost collector can collect and retain household food waste for composting and then be removed when full.

### FIELD OF THE INVENTION

The present invention relates to containers for capturing and temporarily storing compostable material. More specifically, the present invention relates to a household compost receptacle adapted to receive a disposable newspaper liner.

### BACKGROUND OF THE INVENTION

With concerns of landfill space, the need to recycle and compost is becoming more and more important. In particular, for waste disposal, the separation of compostable material from non-compostable material is becoming mandatory in many municipalities. It is common for such municipalities to use what is known as a “Green Bin Program”. Green bin programs often provide a kitchen collector, which is a plastic container that is to be kept in the home to capture the compostable waste material when, where, and as it is produced. The compostable material is then emptied into the larger plastic green bin, on about a daily basis. The green bin is often kept in a garage, or outside, and not in a kitchen, and is put at the curb for pick up by the municipality. Green bin pick-up is often on a weekly schedule, not daily.

A problem is that some municipalities report difficulties with compliance. Compostable materials include such things as organic waste from plant or animal sources. Many people find the collection and storing of this type of material until the weekly pick up day to be dirty, unhygienic and malodorous. The kitchen collector itself captures and stores compostable material for a day or two in the home, most commonly in the kitchen. An unlined kitchen collector will need washing or at least rinsing every time it is emptied. An unlined and unventilated green bin will also get quite dirty as it holds kitchen food waste for up to a week. Compost will begin to decompose and smell even faster in warmer weather, in a closed plastic bin. Any wet remains left in the green bin after pick-up are particularly malodourous, and hosing and cleaning the green bin then becomes a necessary and unpleasant task. An additional problem with wet food material, in winter, is that it will freeze and stick to the green bin, and will then not fall out during attempted collection.

Some municipalities will allow residents to line one or both the kitchen collector or the green bin with plastic bags. Any benefit to the resident is countered by higher costs, as the first task of the municipality is to rip open the bags with extra costly equipment, and then dispose of the bags in landfill. Putting more plastic bags into landfill goes against the purpose of a composting program. Other municipalities that prohibit plastic bags will allow residents to use specialty paper based liners, but residents are required to purchase these. These purchased liners have a short useful life before they are turned into compost.

Used newspaper is a viable paper based liner than can be used in the kitchen collector, and is a recommended liner for both kitchen collectors and green bins by certain municipalities. But loose newspaper is not easy to use to line either the kitchen collector or green bin. The city of Ottawa, Canada provides a step by step guide on how to fold newspaper pages into a liner for the kitchen collector. Some will judge the steps to create the newspaper liner to be complex and cumbersome, and the finished folded liner is not a very good fit with the deep rectangular shape of the plastic kitchen collector used by the city of Ottawa. Such a system will not help encourage more residents to comply with the “green bin program”. Indeed, the instruction pamphlet refers to “origami” and requires the starting paper to be square shaped, which typically requires cutting or tearing of the paper.

### SUMMARY OF THE INVENTION

Disclosed is a compost receptacle that is kept in a home (most commonly in the kitchen) to capture and store compostable material before it is transferred to the green bin outside or other external disposal. The receptacle has a cone shaped opening advantageously adapted to receive a simple newspaper liner. An already folded normal section of a newspaper is dropped, with the folds down, into the receptacle opening. The liner is instantly formed by fanning apart the open adjoining edges of the folded newspaper. The upward facing cone is a simple natural shape for a newspaper to form, and the receptacle is designed as a complementary copy of that shape. It is preferable to use multiple newspaper sheets, so that the liner has sufficient thickness for both strength and absorbency.

Advantageously, many different sizes of newspapers can be used and conveniently constructed into an appropriately shaped liner.

In one embodiment, the receptacle has an accompanying loosely fitted lid, which is dropped onto the newspaper liner. The edges of the lid match the shape of the receptacle, and will trap and seal a newspaper liner of any thickness. The seal is important to stop the ingress or egress of any flies or insects. In one embodiment, the lid has screened openings or other similar provisions for ventilation, and the receptacle itself is also provided with ventilation openings. Ventilation of the liner and the food waste is a very important feature that will be highlighted in accordance with the teachings of the present invention.

When the cone-shaped receptacle is sufficiently full of compost, on about a daily basis, the newspaper liner can be removed, and the upward edges of the newspaper can be folded to produce a sealed bundle. When a newspaper liner is formed in accordance with the teachings of this invention, there are two tall, upwardly extending front and back pointed flaps on the liner, and two shorter edges. The flaps and edges are preferably folded in the order: tall front, short side, short side, and tall back. The last tall flap may be tucked and retained under the two side folds. This bundle is sealed for food and flies, but the newspaper material itself has the important ability to transmit moisture, and the bundle can lose 2% or more of its food weight in evaporated moisture every day.

The inventor has found that where a family of four may produce 10 pounds of food waste in a week, proper ventilation will keep the food waste dry and it will remain odour-free. A further advantage of proper ventilation is that on the weekly collection day, it has typically been found that the food waste will only weigh 9 pounds, and a pound of moisture will have been lost into the atmosphere. This is a pound of liquid that does not need to be cleaned from the green bin, and a 10% weight savings for the municipal collection vehicle. Weight saving translates directly to fuel and cost savings.

Thus, according to one aspect, the invention provides in combination a compost receptacle and removable newspaper liner. The compost receptacle defines an opening therein, the opening comprising two side walls that join together at two wall angles on opposite ends to form a flattened cone shape, the side walls tapering into a point. The newspaper liner is arranged into a shape that is complementary to the shape of the opening with side surfaces that taper into a closed point so that when the opening receives the newspaper liner, the point of the opening receives the closed point of the liner, such that during use the newspaper liner forms a disposable compost collector out of the newspaper liner. In this way, the compost collector can collect and retain household food waste for composting and then be removed when full.

In another embodiment, the invention provides a compost receptacle, comprising an opening defined therein, the opening being in the form of a cone shape and being sized to receive a paper based liner that has been arranged into a complementary cone shape. In this way, food waste can be collected and retained within the cone shaped liner during use. In one embodiment, the cone shaped opening comprises two side walls that join together at two wall angles on opposite ends and taper into a point to form a flattened cone shape. The receptacle may comprise a base for support and/or a lid. Preferably, the receptacle and/or the lid includes ventilation holes. The receptacle may comprise a hanger so that the compost receptacle can be mounted to a door or cabinet. The receptacle may comprise handles cut out from the body of the receptacle.

In another embodiment, the invention provides a method of collecting household compost for recycling. A compost receptacle having a cone shaped opening is used. A paper based liner is constructed into a cone shape that complements the shape of the opening. The paper based liner is placed in the opening to form a removable compost collector. Household compost can be placed in the compost collector. And the compost collector is removed when full of compost. When the compost collector is removed, optionally, upwardly extending flaps of the compost collector can be folded over to form a sealed bundle. The sealed bundle may be placed on a drying rack.

Other aspects and advantages of embodiments of the invention will be readily apparent to those ordinarily skilled in the art upon a review of the following description.

### BRIEF DESCRIPTION OF THE DRAWINGS

Embodiments of the invention will now be described in conjunction with the accompanying drawings, wherein:

FIG. 1 illustrates one embodiment of a compost receptacle and lid in accordance with the teachings of this invention;

FIG. 2 is a top view of the compost receptacle of FIG. 1 with the lid removed;

FIG. 3 illustrates the compost receptacle of FIG. 1 with a newspaper liner in accordance with the teachings of this invention;

FIG. 4 is an unassembled view of the compost receptacle of FIG. 3;

FIG. 5 illustrates the compost receptacle of FIG. 3 with one flap of the newspaper liner folded downwards:

FIG. 6 illustrates a compost receptacle in accordance with the teachings of this invention with a hanger;

FIG. 7 is the cone shaped receptacle of FIG. 6 mounted to a cupboard door;

FIGS. 8a, 8b, 8c and 8d illustrate a perspective view, top view, side view and front view of a drying rack in accordance with the teachings of this invention;

FIGS. 9a and 9b illustrate top views of possible newspaper liners when constructed;

FIGS. 10, 11a, 11b and 11c illustrate how to form a newspaper liner for the cone shaped receptacle of FIG. 1;

FIG. 12 illustrates a newspaper liner in accordance with the teachings of this invention, which is filled with compost and folded to form a sealed bundle;

FIG. 13 illustrates one method of drying bundles of FIG. 12, in accordance with the teachings of this invention; and

FIG. 14 illustrates another method of drying bundles of FIG. 12, in accordance with the teachings of this invention.

This invention will now be described in detail with respect to certain specific representative embodiments thereof, the materials, apparatus and process steps being understood as examples that are intended to be illustrative only. In particular, the invention is not intended to be limited to the methods, materials, conditions, process parameters, apparatus and the like specifically recited herein.

### DETAILED DESCRIPTION OF THE DISCLOSED EMBODIMENTS

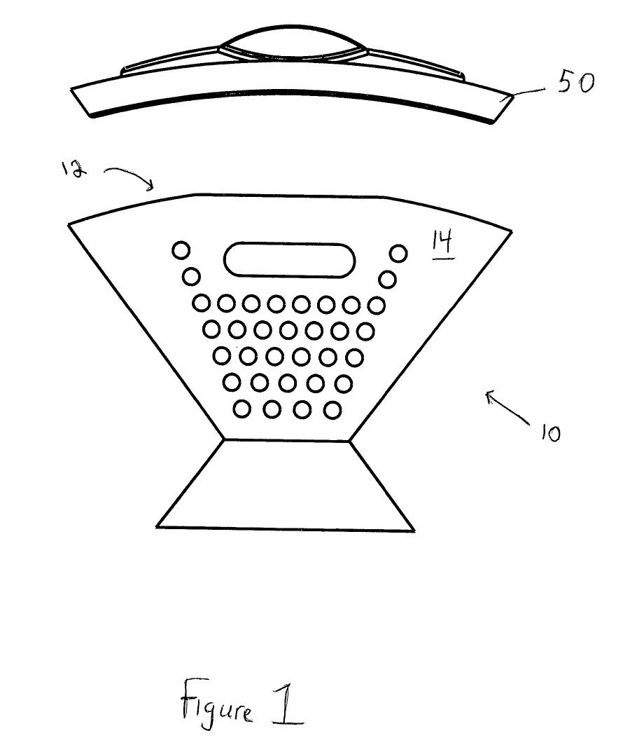

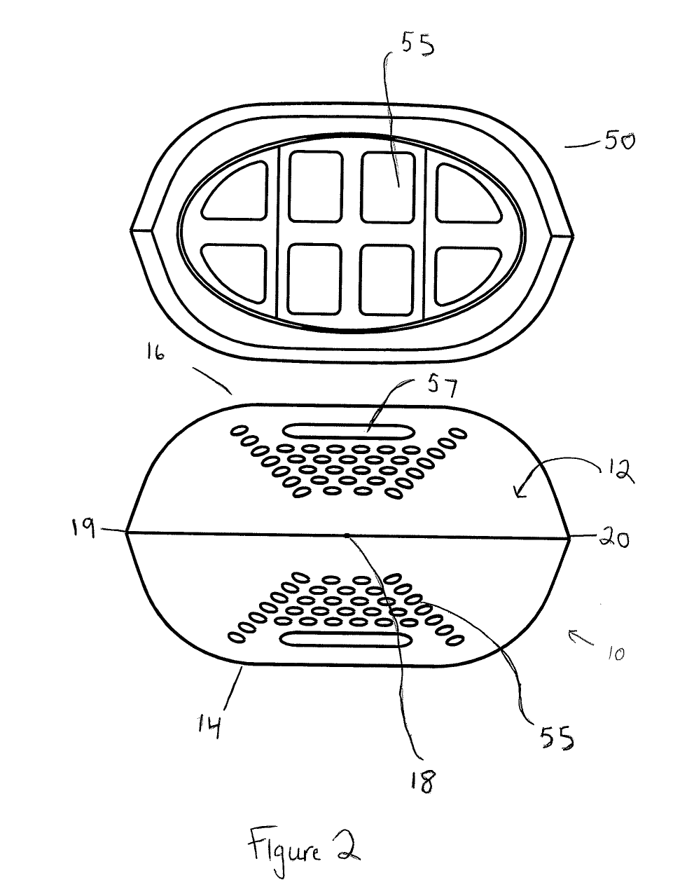

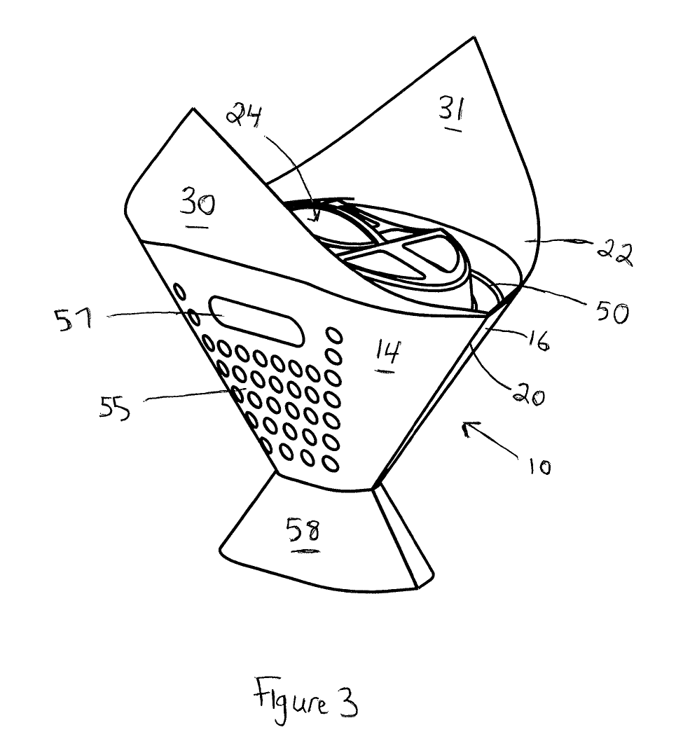

Referring to FIGS. 1 and 2, there is shown a compost receptacle 10 adapted to receive a removable newspaper liner (seen in FIG. 3) in accordance with the teachings of this invention. The compost receptacle 10 comprises a cone shaped opening 12 therein with two side walls 14, 16 that taper to a point 18. Preferably the opening 12 is shaped as a flattened cone with the two side walls 14, 16 joining together at two wall angles 19, 20 on opposite ends to form a flattened cone shape. The result is a top profile that is similar to an elongated football shape (seen in FIG. 2). For example only, overall dimensions of the a compost receptacle 10 in accordance with the teachings of this invention are 9.5″ (24 cm) tall, 13″ (33 cm) wide, and 7″ (18 cm) deep.

Referring to FIG. 3, the newspaper liner 22 is arranged into a cone shape complementary to the shape of the opening 12, such that the opening 12 can receive and support the liner 22 therein during use to form a disposable compost collector 24 out of the newspaper liner 22. The compost collector 24 can collect and retain household compost and then be removed when full. In this way, the compost receptacle 10 stays clean as the compost is contained within the disposable compost collector 24, and the outdoor green bin stays clean as well. As a further feature to facilitate cleaning at the lowest point 18, the receptacle walls can be locally spread open to a rounded shape that will admit the width of a finger tip. In this embodiment, the overall shape of the receptacle is still based on the presence of corner 18, and this opened shape does not interfere with the functioning of the corner 18.

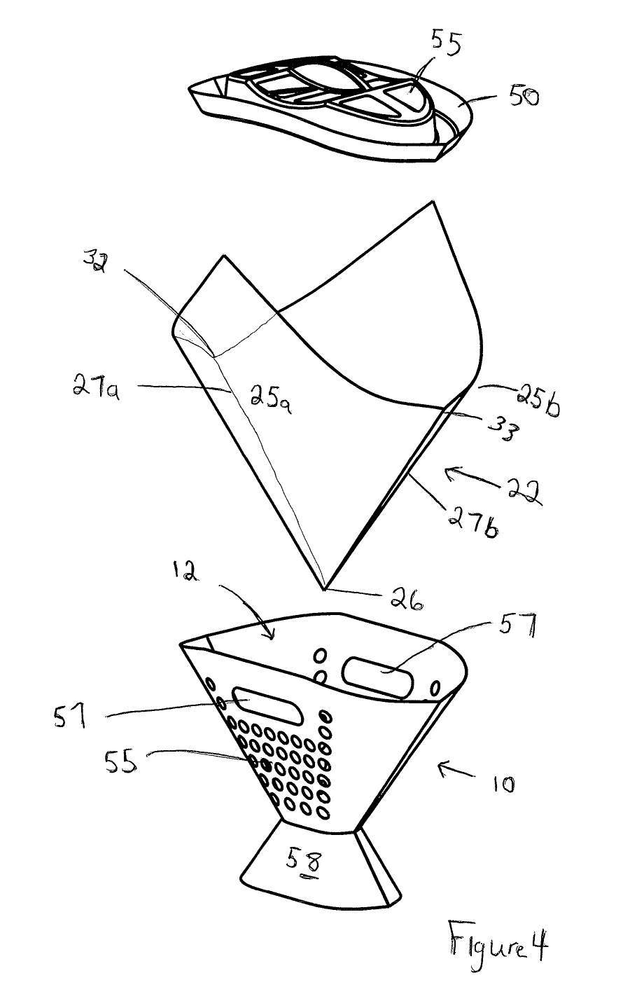

The cone shaped opening 12 allows the user to use a liner 22 that is easily created in a complementary shape out of newspaper (seen in FIG. 4). Since the opening 12 is a cone shape, a folded newspaper section is easily dropped in and the open adjacent edges fanned apart. This newspaper liner can be opened to either a flattened cone shape or a fully circular cone shape. This is described in detail below. This creates a compost collector 24 with side surfaces 25a, 25b that taper to a closed point 26. This closed point 26 aligns with the point 18 of the opening 12. Preferably, the newspaper liner is folded as a flattened cone shape with side surfaces 25a, 25b that meet at opposite angles portions 27a, 27b. This flattened cone shaped compost collector 24 self-aligns within and is supported by the opening 12 of the compost receptacle 10, and household compost can easily be collected therein. If more than a single sheet of newspaper is used, the liner becomes stronger, thicker and more absorbent.

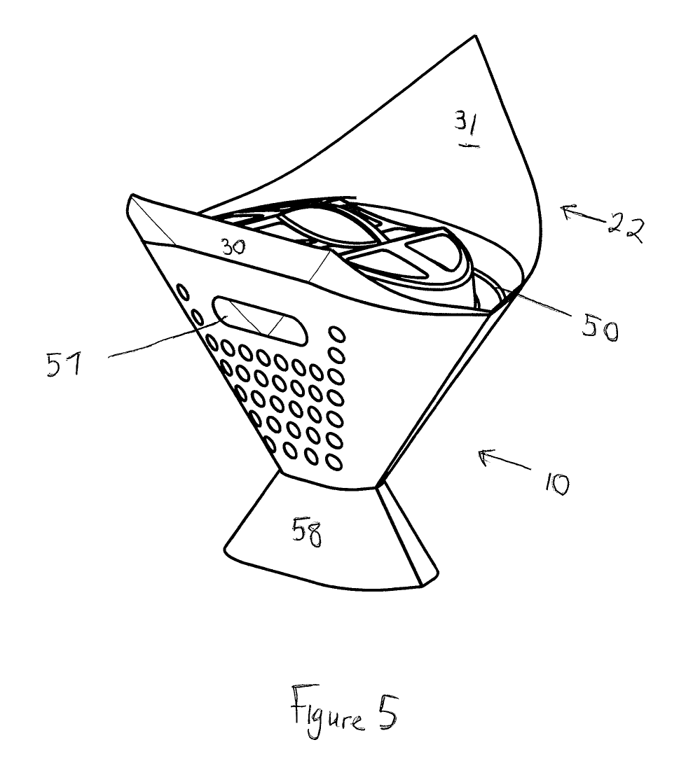

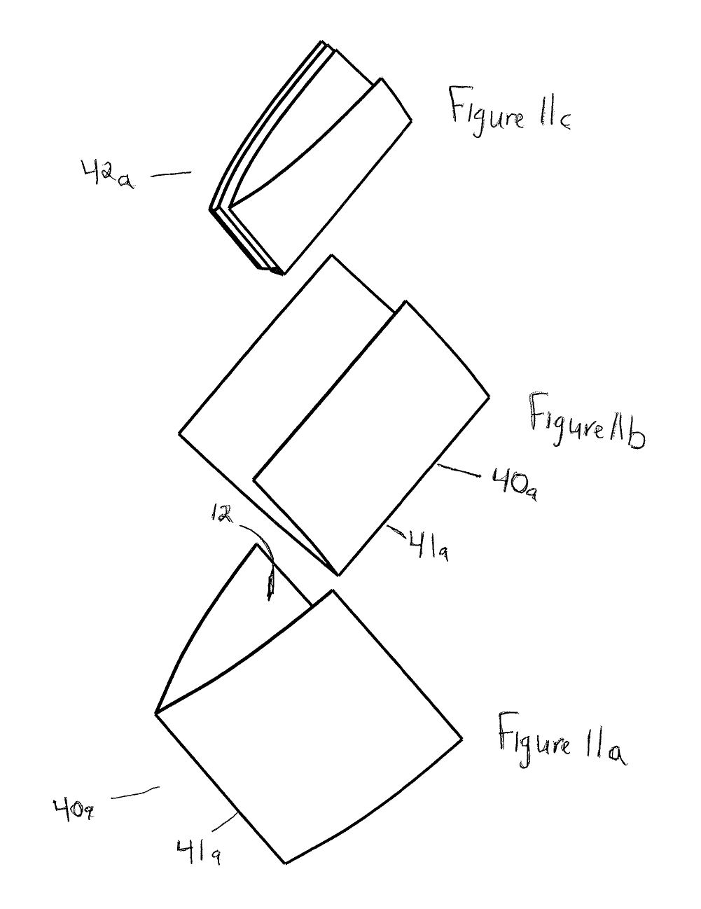

Referring to FIGS. 3, 4 and 5, when the newspaper is deposited and held in accordance with the teachings of this invention, the compost collector 24 inherently includes upwardly extending flaps 30, 31, and lowest corners 32 and 33. When the compost collector 24 is sufficiently full, but not over full, it may be removed from the opening 12. For removal, loosely fold in flap 30, and remove the collector 24 to a convenient counter-top. Complete the folding by tightly folding the flaps in the order 30 (tall), 32 (short), 33 (short) and 31 (tall). With care, the last tall flap 31 can be folded over and tucked back underneath the two short side flaps 32, 33, to form a sealed bundle 39, as seen in FIG. 12. Alternative sealing methods are to use a small piece of compostable adhesive backed paper tape (masking tape) on the final fold, or to leave the bundle loosely folded.

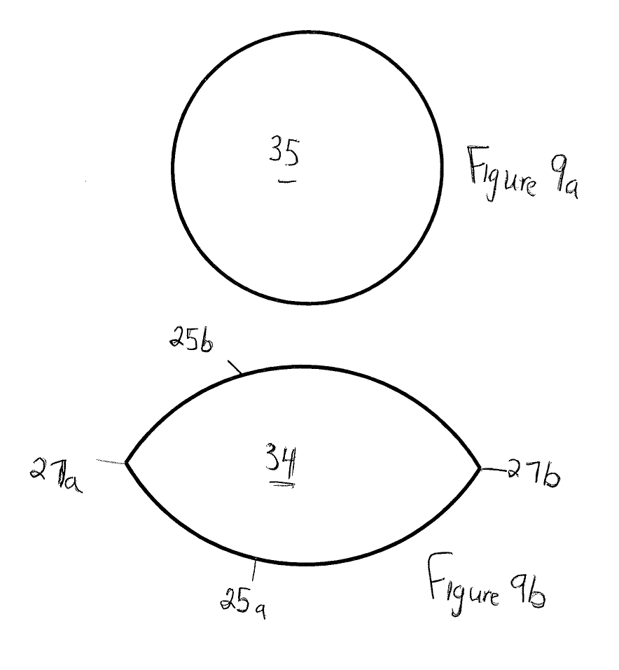

Referring to FIGS. 9a and 9b, the newspaper liner can be opened anywhere from flat (not shown), to a partly open cross-section shape 34 (FIG. 9b) to a fully opened circular cross-section shape 35 (FIG. 9a), but the full circle 35 does not have the corner angles 27a, 27b which tend to align the newspaper cone 22. The preferred aspect ratio is in the range around 1.8:1 to 2:1 This results in an opened cone or compost collector 24, which will hold a volume of compost, but this “football” shape or flattened cone shape retains the angles 27a, 27b where the curved surfaces 25a, 25b meet. The folded edges 27a, 27b of the newspaper liner 22 will self-align into the corners 19, 20 of receptacle 10. The corners are preferably no more than about 145 degrees. The final curved surfaces 25a, 25b can be complex (splines), and are not restricted to simple conical curves. In the flattened state, as in a flattened newspaper, surfaces 25a and 25b would begin with a square lower corner, i.e. edges 27a and 27b would be at right angles (90 degrees) at corner 26. As the shape is opened, the angle begins to reduce from 90 degrees. In the preferred embodiment, in the actual final curved state, edges 27a and 27b form an angle of about 74 degrees.

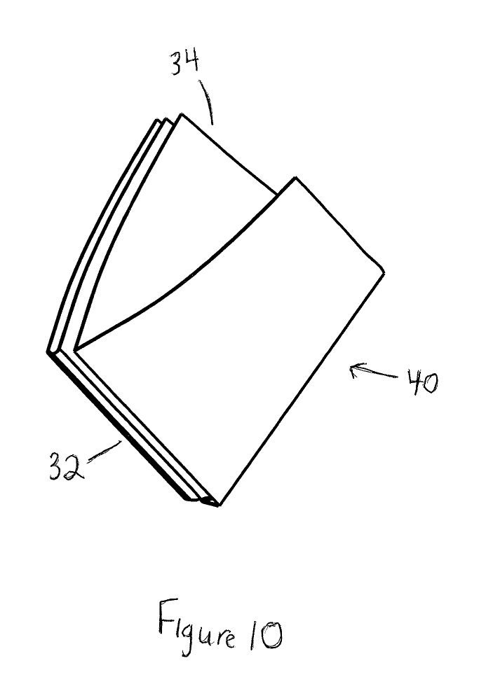

Referring to FIGS. 10, 11a, 11b and 11c, there are illustrated methods to construct a newspaper liner in accordance with the teachings of this invention. It should be understood that the word “newspaper” used within this application refers to any paper used in a publication such as a newspaper, tabloid, bulletin, gazette and the like, being of various sizes.

FIG. 10 illustrates how to make a broadsheet newspaper 40 into a newspaper liner 22. This is the preferred method, which uses broadsheet newspapers 40 that are typically already folded twice. The first fold is on the normally vertical axis, forming pages, and the second fold, horizontally, is for compactness and presentation. A doubly-folded section of newspaper (usually 3 or more sheets, 12 or more pages) is aligned and simply dropped into the opening 12 of the receptacle 10. All of the folded edges 32 are to the bottom, and all of the unfolded edges face upward 34. The liner cone is formed by opening the section at one of the normally vertical page folds. This results in about ¼ of all the sheets moving in one direction, and the remaining ¾ of the sheets moving in the opposite direction.

Broadsheet newspapers are typically sheets around 22 to 23″ tall, by 23″ wide. Folded once to about 22-23″×11½, folded twice to 11½×11 1/2.

FIGS. 11a, 11b, and 11c illustrates how to use a tabloid sized newspaper 40a Tabloid size newspapers are typically single section papers, with a single normally vertical fold 41a. To use this format, the user first places one or two folded sheets 40a into the holder with the fold 41a down, and to one side of the opening 12 (FIG. 11a). The fold 41a will line up with one of the angled corners 19 or 20 of the opening 12. Then the user places one or two more folded sheets 40a into the opposite side of the opening 12, with the fold down 41a and lined up with the other angled corner 19 or 20 of the holder 10 (FIG. 11b). Then the user double folds another one or two folded sheets 40a to make it look like a small version of the previous method's FIG. 10 (FIG. 11c). This last folded sheet 42a is then dropped into the opening 12 of the receptacle 10 to cover the bottom point 26.

Tabloid papers are typically sheets around 12½″ tall, by 23″ wide. The sheets are folded once vertically to make a paper 12½″ tall by 11½″ wide.

Embodiments of the invention contemplate use of other sizes of newspaper to create a liner. For example, other sizes can use a modified version of either above method. For instance, a taller tabloid paper, around 15 to 16½″ tall by 11½″ wide, can use a method similar to the first method used for broadsheet paper. For the taller tabloid, add a horizontal fold, not in the middle of the paper, but closer to the bottom. The finished newspaper liner has a long side of the fold which is about 11½″, and the short side will only be 3½ to 5″ long. The newspaper liner is dropped into the opening and the cone is formed by opening the long side of the folded sheets.

As can be seen, preferably, the compost receptacle includes a lid 50. The lid can serve to close the receptacle 10 and prevent the entrance or exit of flies and insects, alleviating such problems as fruit flies. The lid 50 has an outer edge shape which matches the opening 12. Any thickness of liner 22 will be trapped and sealed by lid 50. In a preferred embodiment, the receptacle 10 and/or the lid 50 includes areas of screening or other ventilation 55. Such areas 55 are preferred for the benefits of ventilation and drying of the food waste. The screened openings 55 allow for the slow diffusion of moisture from the food waste to the outside environment while not allowing large air flows that may carry odours.

The main body of the receptacle also preferably has ventilation holes 56. These holes may be larger (¼″ to ½″) and need not be screened. They are also important for ventilation and the removal of moisture from the food waste. The newspaper material of the compost collector 24 will absorb moisture, and the holes 56 will allow some of that moisture to evaporate and be diffused into the outer environment. These holes need not be screened because the newspaper compost collector 24, by itself prevents any direct air or insect access to the food waste.

In one embodiment, the compost receptacle may also include handles 57 cut out in the body of receptacle 10. The compost receptacle includes a base portion 58 so that the receptacle 10 can be free standing on a surface, such as a kitchen counter.

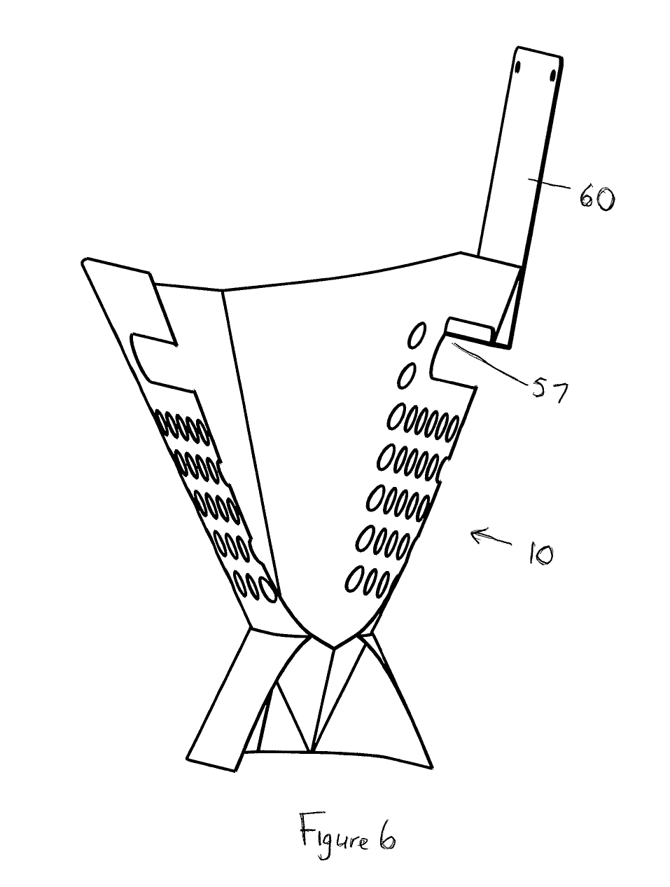

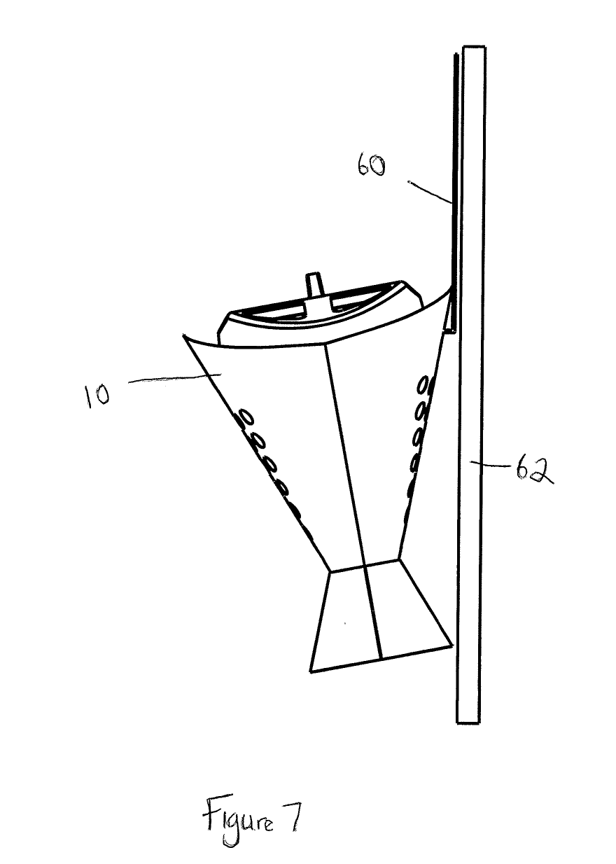

Referring to FIGS. 6 and 7, in one embodiment, the invention also contemplates use of a hanger 60 to support the body of the compost receptacle 10 so that the compost receptacle 10 can be mounted to a door or cabinet 62. This allows the user the choice between keeping the receptacle 10 for instance on the kitchen counter or hanging on the interior of a cabinet door 62. In the embodiment shown, the hanger 60 supports the compost receptacle 10 through the handle opening 57.

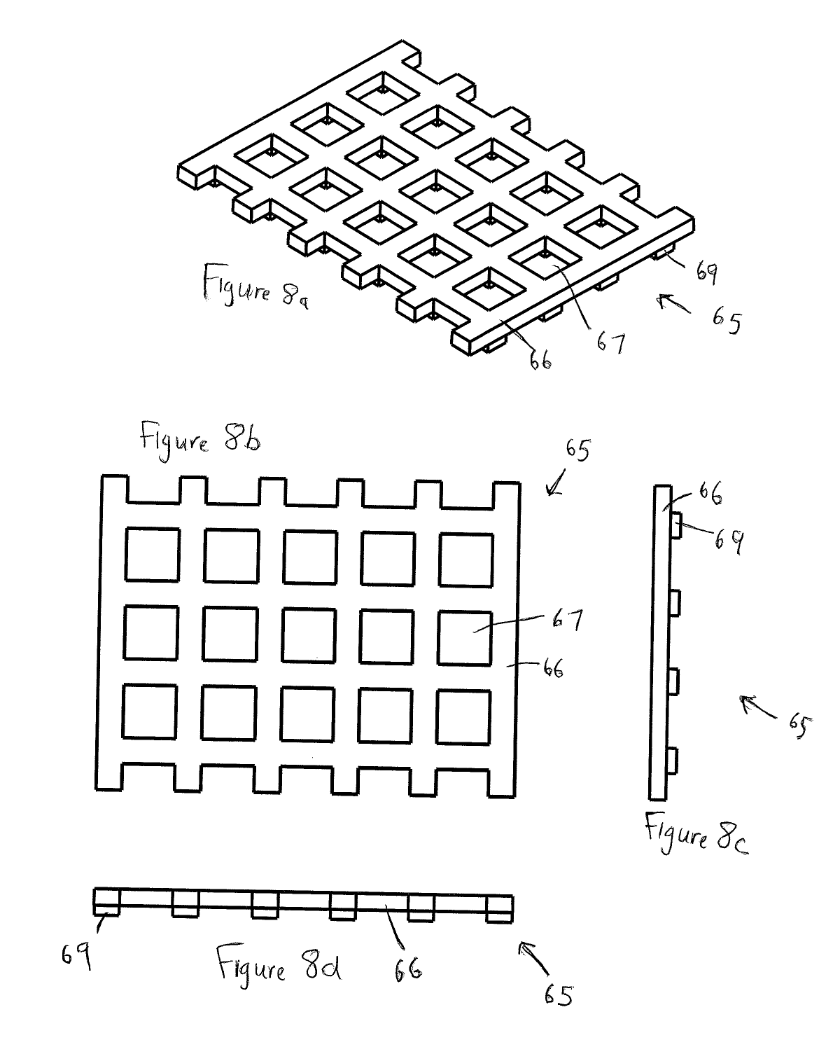

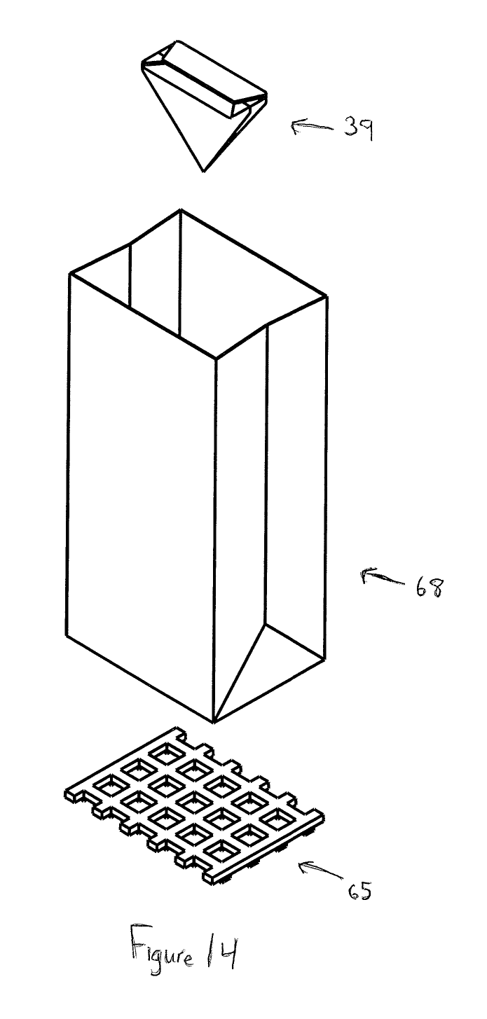

Referring to FIGS. 8a, 8b, 8c and 8d, in one embodiment, the invention also contemplates use of a drying rack 65 adapted to receive multiple compost collection bundles 39. As illustrated, the drying rack 65 comprises a frame 66 with multiple ventilation slots 67 defined therein. The drying rack 65 is raised via feet 69. The drying rack 65 is preferably sized about 12″×16″, to support and ventilate the bottom of a large Kraft-paper bag 68 (FIG. 14). These bags 68 are commonly sold to collect yard and garden waste.

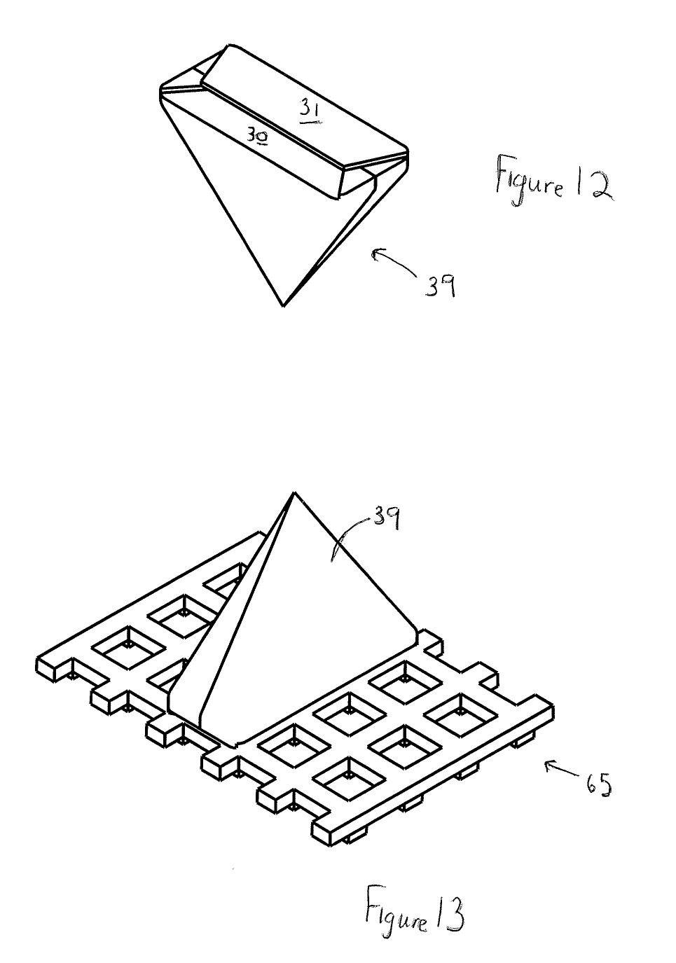

The drying rack 65 can be used in a garage or out-building in one of two ways. As seen in FIG. 13, the drying rack 65 may be used to hold multiple individual full bundles 39, to allow ventilation until they can be disposed of. Alternatively, as seen in FIG. 14, drying rack 65 may also be used to support a yard-waste paper bag 68, into which multiple bundles 39 are placed. If the bundles 39 are only loosely bundled or sealed, then the top of the yard-waste bag 68 should be folded closed to prevent any insect problems. The paper material of the bag 68 will allow continued diffusion of moisture through it to the atmosphere, in the days leading up to municipal pick up.

Either of these drying methods can be used in order to dry the food waste as much as possible prior to outside disposal. Storing food waste for days in a sealed plastic green bin allows for no escape of moisture, and the wet material smells much worse than when it is allowed to dry.

Just prior to pick-up, the compost collection bundles 39 are finally placed into the green bin, to be taken to the curb. As noted, the conventional green bin does not allow for proper ventilation, and using a sealed plastic bin for days and days of storage will only lead to problems with excess moisture and foul smells. On the other hand, because it is sealed, it is very good at preventing animals from getting at the food waste, and the use of the green bin is the preferred final step in this method for municipal curb-side collection.

The method of collecting household compost for recycling in accordance with the teachings of this invention is now described. A compost receptacle 10 having an opening 12 therein as described above can be used. The receptacle 10 can be located in any convenient location for the user. For instance, the receptacle can either be placed on the counter or mounted on the interior of a cabinet door.

As needed, the user can easily construct a paper based liner 22 into a cone shape that complements the shape of the opening 12. A method of constructing the liner 22 is described above. The paper based liner 22 is then placed in the opening 12 of the receptacle 10 to form a removable compost collector 24.

During use, household compost can be collected within the compost collector 24, thus keeping the compost receptacle 10 clean. The mouth of the opening 12 is very large, allowing for the easy scraping of food waste from dinner plates, etc. At any time, or when the compost collector 24 is full, it can be removed from the opening 12 and a new liner 22 can be placed in the compost receptacle 10. Unlike the use of the costly cellulose lined paper bags, the newspaper compost collection liner can be changed anytime and as often as you wish, at no cost.

The flaps 30, 31, 32, 33 on the compost collector 24 can be folded over to form a sealed bundle 39. The bundle 39 should not be placed directly into the green bin if scheduled pick-up is many days away. It can be stored on a drying rack 65, by itself, or inside a paper yard waste bag, before disposal, for instance by municipal pick up.

When the/each compost collector 24 is placed in a green bin or other container for disposal from the home, that container is also kept clean as only the outside of the newspaper bundle ever touches the inside of the green bin. The food waste itself never touches the green bin.

Embodiments of the invention have been described using a liner of newspaper. However any suitable paper based liner can be used. For instance, sheets of craft paper can be used in place of newspaper. Newspaper is preferable, as it is readily available and in many cases, already needing to be disposed of.

Numerous modifications may be made without departing from the spirit and scope of the invention as defined in the appended claims.

What is claimed is:

1. In combination a compost receptacle and removable newspaper liner, wherein:
the compost receptacle defines an opening therein, the opening comprising two side walls that join together at two wall angles on opposite ends to form a flattened cone shape, the side walls tapering into a point; and
the newspaper liner is arranged into a shape that is complementary to the shape of the opening with side surfaces that taper into a closed point so that when the opening receives the newspaper liner, the point of the opening receives the closed point of the liner, such that during use the newspaper liner forms a disposable compost collector out of the newspaper liner;
whereby the compost collector can collect and retain household food waste for composting and then be removed when full.

2. The combination of claim 1, wherein the newspaper liner is shaped as a flattened cone such that the side surfaces meet at opposite angle portions that self-align with the two walls angles of the opening when the opening receives the liner.

3. The combination of claim 2, wherein the compost collector comprises more than one sheet of newspaper.

4. The combination of claim 2, wherein the receptacle has a base to support the receptacle in a generally vertical free standing position.

5. The combination of claim 2, wherein the receptacle further comprises a lid.

6. The combination of claim 5, wherein the receptacle and/or the lid includes ventilation holes.

7. The combination of claim 2, wherein the compost collector includes an upwardly extending flap(s) which can be folded over to cover the compost collector when the compost collector is full and is to be removed from the opening to form a sealed bundle.

8. The combination of claim 7, further comprising a drying rack adapted to receive multiple sealed bundles for drying prior to disposal.

9. The combination of claim 2, further comprising a hanger for the body of the compost receptacle so that the compost receptacle can be mounted to a door or cabinet.

10. A compost receptacle, comprising:
an opening defined therein, the opening being in the form of a cone shape and being sized to receive a paper based liner that has been arranged into a complementary cone shape;
whereby food waste can be collected and retained within the cone shaped liner during use.

11. The compost receptacle of claim 10, wherein the cone shaped opening comprises two side walls that join together at two wall angles on opposite ends and taper into a point to form a flattened cone shape.

12. The compost receptacle of claim 11, further comprising base for support.

13. The compost receptacle of claim 11, further comprising a lid.

14. The compost receptacle of claim 13, wherein the receptacle and/or the lid includes ventilation holes.

15. The compost receptacle of claim 11, further comprising a hanger so that the compost receptacle can be mounted to a door or cabinet.

16. The compost receptacle of claim 11, further comprising handles cut out from the body of the receptacle.

17. A method of collecting household compost for recycling, comprising:
using a compost receptacle having a cone shaped opening;
constructing a paper based liner into a cone shape that complements the shape of the opening;
placing the paper based liner in the opening to form a removable compost collector;
placing household compost in the compost collector; and
removing the compost collector when full of compost.

18. The method of claim 17, wherein the opening comprising two side walls that join together at two wall angles on opposite ends and taper into a point to form a flattened cone shape, and the step of constructing a paper based liner into a cone shape that complements the shape of the opening comprises:
folding a sheet of newspaper to define two sides surfaces that join together at opposite angle portions, and
fanning apart two adjoining open corners, resulting in the side surfaces tapering to a closed point;
whereby the newspaper liner self-aligns within the opening of the compost receptacle.

20. The method of claim 19, wherein the step of removing the compost collector when full of compost comprises folding over upwardly extending flaps of the compost collector to form a sealed bundle.

21. The method of claim 20, further comprising the step of placing the sealed bundle on a drying rack.
